(19) United States
(12) Patent Application Publication
(10) Pub. No.: US 2010/0095977 A1
(43) Pub. Date: Apr. 22, 2010

(54) HAIR BAND WITH RETRACTABLE EYE WEAR AND REMOVABLE DECORATIVE COVER

(76) Inventors: Paul Schmitz, Huntersville, NC (US); Gary Greene, Matthews, NC (US)

Correspondence Address: SUMMA, ADDITON & ASHE, P.A., 11610 NORTH COMMUNITY HOUSE ROAD, SUITE 200, CHARLOTTE, NC, 28277, US

(21) Appl. No.: 12/580,521

(22) Filed: Oct. 16, 2009

Related U.S. Application Data
(60) Provisional application No. 61/105,827, filed on Oct. 16, 2008.
(60) Provisional application No. 61/159,932, filed on Mar. 13, 2009.

(51) Int. Cl. A45D 8/12; A61F 9/00; A42B 1/24

(52) U.S. Cl. 132/275; 2/15; 2/209.14

(57) ABSTRACT

A hair band assembly includes a lower band element and an upper band element defining a cavity there between. A pair of eyeglasses, including a frame element enclosing lenses therein, is connected to the hair band assembly via a connector that allows the eyeglasses to pivot between two positions an extended position on the individual's face, and a refracted position within the cavity of the hair band. The frame element on the eyeglasses includes a tab that projects outwardly from the side of the eyeglasses. The tab fits into a recess defined within the upper band element to allow easy retrieval. The frame element also includes a protrusion that mates with a recess on the lower side of the upper band element, such that the recess forms a protrusion snap lock for holding the eyeglasses in place within the cavity. The hair band assembly encompasses a reversible decorative element that is removable from the hair band. The decorative element includes patterned fabric on either side and is attachable to the hair band by a pocket tab. The pocket tab is adjustable for flipping inside out to display one side of fabric or the other on the same hair band.

### CROSS REFERENCE TO RELATED APPLICATIONS

This application claims the benefit of prior co-pending U.S. Provisional Patent Application Ser. No. 61/105,827 filed on Oct. 16, 2008. This prior application is incorporated by reference herein.

This application also claims the benefit of prior co-pending U.S. Provisional Patent Application Ser. No. 61/159,932 filed on Mar. 13, 2009. This prior application is incorporated by reference herein.

### FIELD OF INVENTION

The application relates to the field of hair bands with additional accessories including retractable eyeglasses or sunglasses and decorative removable covers thereon.

### BACKGROUND

Many women and children use decorative hair bands as fashion accessories. Several hair band designs allow for interchangeable decorations making the hair band adaptable for varying occasions and tastes. For example, U.S. Pat. No. 7,156,106 to Lawson discloses a foldable decorative hair band. The hair band in Lawson has a rail strip whereby various decorative plates are slidably attached to or removed from the hair band. U.S. Pat. No. 6,688,316 to Klug also discloses a hair band having interchangeable decorations. Decorations are attached and removed by using hook and loop fasteners.

Sun glasses have also become a popular accessory not just for their practical value in protecting the eye from harmful solar rays but also for ornamental and fashion value. Because of the size of glasses and their effect of reducing visible light seen by their wearers, sunglass wearers commonly remove them to improve their vision and physical comfort. Once removed, sunglasses wearers have the burden of keeping track of their sunglasses. For the convenience of sunglasses wearers, efforts have been made to combine sunglasses with various types of headgear. The prior art typically shows eyeglasses in combination with certain kinds of headbands that extend across an individual's forehead. For purposes herein, therefore, a “headband” is construed to extend around at least a portion of the front of the head (e.g., the forehead). In contrast, a “hair band” at least partially engages the top of the head (i.e., the region on the skull where hair typically grows to a length that requires control and keeping in place).

U.S. Pat. No. 4,712,254 to Daigle discloses a headband and eyeglasses combination. The headband is made from a flexible and resilient cloth and includes a pocket therein that extends across the forehead when the headband is worn. Eyeglasses are attached to the headband with a mounting assembly. The mounting assembly includes track elements for sliding the eyeglasses between a refracted position within the pocket of the headband and a downwardly extended operative position when the wearer uses the glasses. When the eyeglasses are retracted, they are positioned entirely within the pocket.

U.S. Pat. No. 5,009,496 to Holtan also discloses an eyeglasses and headgear combination. The eyeglasses frame is attached to a headband by a coupling member. The coupling member includes a pivoting member for pivoting the eyeglasses about an axis. When the eyeglasses are pivoted upwards away from the eyes, the eyeglasses remain visible and not obscured by the headband.

U.S. Pat. No. 5,105,475 to Lynd discloses a head gear element in the general shape of a visor with an eye shield incorporated therein. The head gear element includes a visor cavity within the headband portion, and the eye shield pivots from a position within the cavity to a position on the wearer's face. The forehead section within the head gear element is formed from suitable plastic having front and rear walls interconnected by a top wall defining the visor cavity for storing the eye shield. The eye shield is coupled to the head element with a support bracket. The support bracket enables the eye shield to be pivoted from a position in front of the wearer's eyes to a position within the storage cavity.

Design patents of interest in this field include U.S. Pat. Nos. D331,250 (Acosta 1992) and D338,099 (Gentile 1993).

None of the above-noted prior patents show or suggest eye wear that can be disposed within a hair band worn on top of the head (i.e., in the hair). Such a hair band assembly would be a convenient accessory to accommodate a consumer's desire for fashion and functionality.

### BRIEF SUMMARY OF THE INVENTION

A hair band assembly includes a hair band that defines a cavity therein, and the cavity is accessible from one side of the hair band. The hair band assembly includes eyewear connected to the hair band. The eyewear is connected to the hair band in a pivoting relationship such that the eyewear moves along an arcuate path from an extended position on the wearer's face to a retracted position within the cavity.

The hair band may include an upper band element, wherein the upper band element is shaped as an open loop that conforms to a head of a wearer when positioned over the top of the wearer's head. The hair band further includes a lower band element connected to the upper band element, wherein the lower band element is shaped as an open loop that conforms to the head of the wearer when positioned over the top of the head. The upper band element and the lower band element define a cavity there between. A frame element extends around a wearer's face. A coupling element connects the frame element to at least one of the band elements, wherein the coupling element enables the frame element to be pivoted between an extended position on a wearer's face and a retracted position in the cavity of the hair band.

The hair band also incorporates a removable decorative element that attaches and detaches from the hair band via reversible pocket tabs.

### BRIEF DESCRIPTION OF THE FIGURES

FIG. 1 is a perspective view of a hair band defining a cavity for eyewear to fit within the cavity.

FIG. 2 shows the hair band of FIG. 1 on the head of a wearer with the eyewear in the extended position.

FIG. 3 shows a cross section of the hair band assembly described herein with the eyewear in the extended position.

FIG. 4 shows a cross section of the hair band assembly described herein with the eyewear in the retracted position inside the cavity of the hairband.

FIG. 5 shows a front view of the hair band of FIG. 1.

FIG. 6 shows an up close cross section view of the hair band and the cavity therein.

FIG. 7A shows removable eyewear used with the hair band described herein.

FIG. 7B shows a close up view of the temple portion that allows for snapping the eyewear of FIG. 7A into the hair band of FIG. 1.

FIG. 8 shows the hair band of FIG. 1 adapted for fitting a decorative element thereon via pocket tabs described herein.

FIG. 9 shows the hair band of FIG. 8 with the decorative element affixed to the hair band.

FIG. 10A shows the decorative element connector in the form of a pocket tab on one side of the decorative element.

FIG. 10B shows the wearer pulling the pocket tab to reverse the decorative element and move the pocket tab to the opposite side of the decorative element.

FIG. 10C shows the wearer pulling the pocket tab over the hair band in a way that reverses the pocket tab and displays an opposite side of the decorative element in comparison to FIG. 10A.

FIG. 10D shows the decorative element on the opposite side of the hair band as compared to FIG. 10B.

FIG. 11 shows the hair band described herein with a decorative element affixed thereon by pocket tabs.

FIG. 12 shows the reversible nature of the decorative elements described herein.

### DETAILED DESCRIPTION

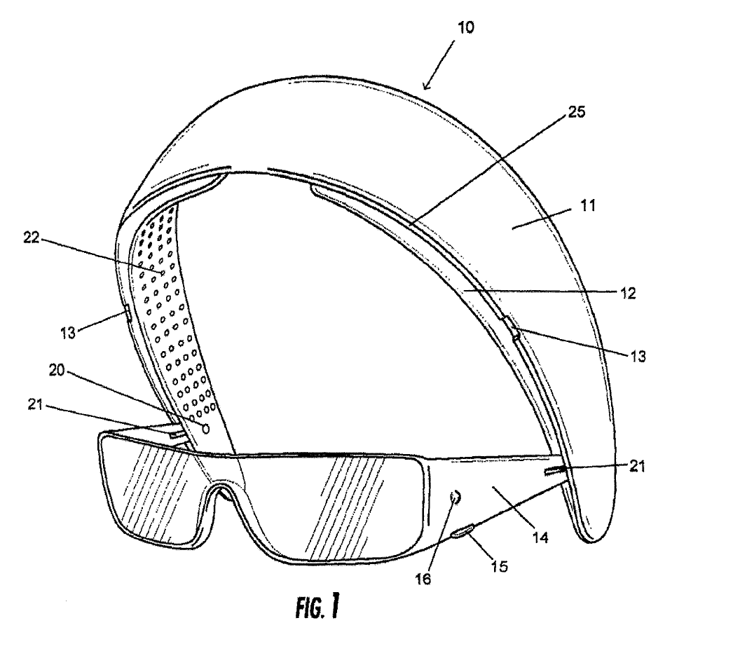

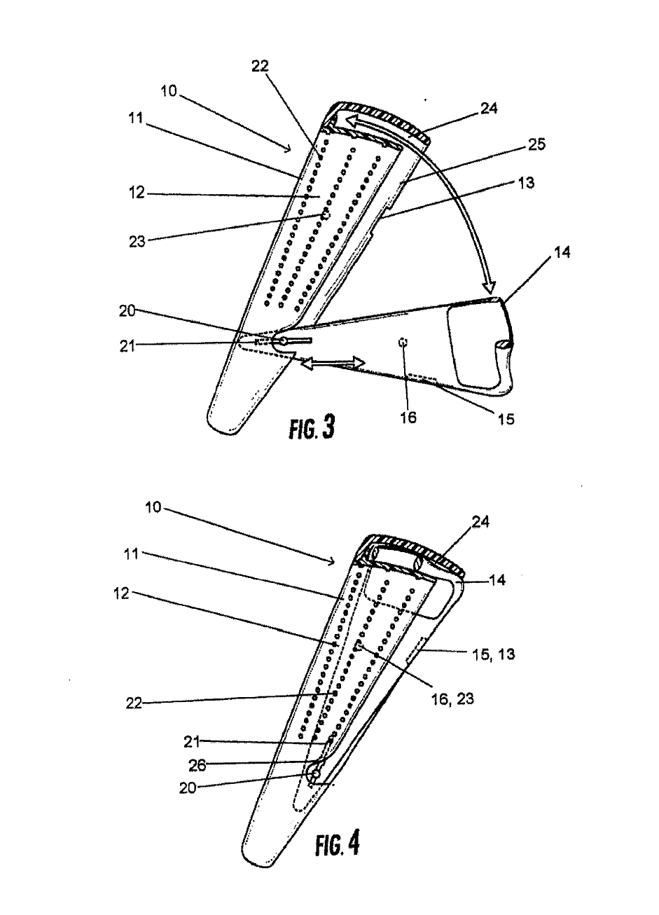

FIG. 1 is a perspective view of one embodiment of the invention and is a hair band structure broadly designated at 10. The hair band 10 has an upper band element 11 and a lower band element 12. In one embodiment, the band elements are formed from a flexible plastic. As depicted in FIG. 1, the band elements 11 and 12 are shaped as respective open loops that conform to the shape of the head of a wearer when positioned over the top of the wearer's head. For purposes herein, a hair band at least partially engages the top of the head. In one embodiment, a hair band defines an opening that fits over an individual's head, and the hair band extends from a first tip portion 32 (see FIG. 5) proximate the wearer's ear, across the top of the head, and continues over the head to a second tip portion 32 proximate the other ear on the opposite side of the head.

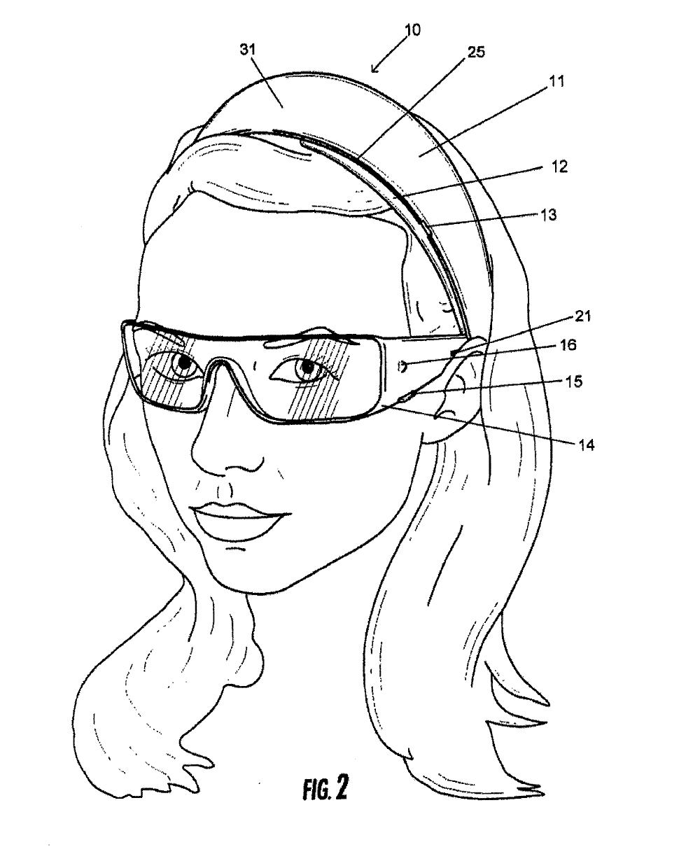

FIG. 2 depicts one of the embodiments of the invention being worn. When worn, the hair band 10 is positioned substantially vertically on the wearer's head so that a top portion 31 of the upper band element 11 is positioned substantially above the top of the wearer's head. The lower band element 12 includes a textured surface 22 which helps to secure the hair band 10 on the wearer's head.

One of the purposes of this invention is to provide a combination including eye wear and a hair accessory. In a preferred embodiment, the eye wear is pivoted from a position on the wearer's face (i.e., the extended position) to a position that is substantially, if not entirely, enclosed within the hair band (i.e., the retracted position). In this regard, the hair band 10 further includes a frame element 14 for holding the lenses of a pair of glasses. The lenses within the frame element 14 can be used for one or more purposes including but not limited to sight correction, safety, sunlight protection, and fashion.

FIG. 3, which is a cross-sectional view of the hair band 10, depicts the frame element 14 in the extended position. This is the position in which the wearer would be using the glasses over the eyes. When in an extended position, therefore, the frame element 14 is positioned in front of the wearer's eyes as a typical pair of glasses. The frame element 14 is mechanically coupled to at least one and possibly both of the band elements 11 and 12 by a coupling element 20. In a preferred embodiment, the upper band element 11 and the lower band element 12 are connected to form the overall single hair band that accommodates retractable eye wear. The upper band element 11 and the lower band element 12 may be connected at the lower tips 32 on either side. Coupling element 20 may be used to connect the upper band element 11, the lower band element 12, and the frame element 14 of the overall hair band 10. Alternatively, the coupling element 20 may connect any two of the band elements 11, 12 and the frame element 14. In one embodiment, the coupling element 20 is a post that fits within a cavity 24 defined by the hair band and engages a portion of the frame element 14 to allow pivoting in extended and retracted positions.

The frame element 14 defines a slide rail 21 (FIGS. 3 and 4), which engages the coupling element 20. In a preferred embodiment, the slide rail 21 is a substantially horizontal opening in the frame element 14, and the slide rail 21 has edges that define an extended oval or rectangle. In one embodiment, the coupling element 20 may include regions of different sizes, such that the coupling element fits within the slide rail 21 but does not slide out. In other words, in the embodiment of FIG. 3, the end of the coupling element 20 is larger than the portion of the coupling element 20 within the slide rail 21.

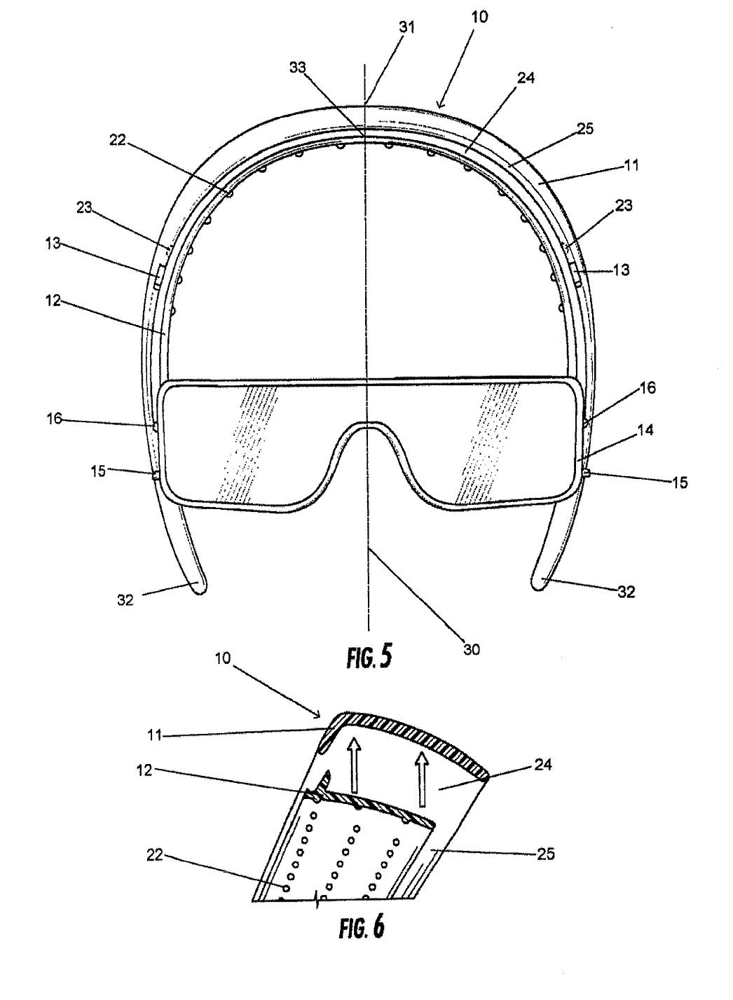

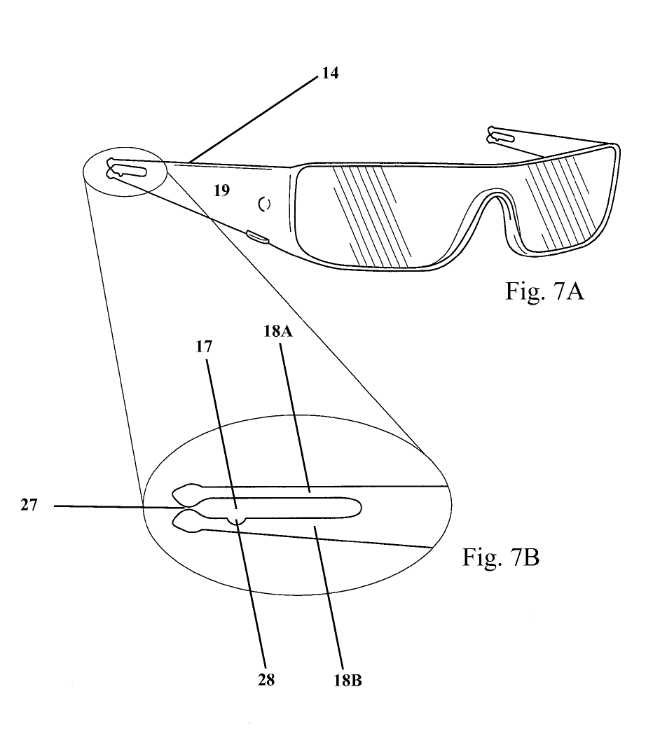

In a different embodiment, the frame element 14 is entirely removable from the hair band 10. In the embodiment of FIG. 7, the temple portion 19 of the frame element 14 defines a groove 17, similar to the above noted slide rail, at a distal end opposite the lens portion of the eyewear. For purposes herein, the term “distal end” is from the perspective that the eyewear lenses would be the most “proximal” portion to a person facing the wearer. The distal end, therefore, is the end of the eyewear that would typically be positioned adjacent the wearer's ear. The temple portion 19 terminates at the distal end in two legs 18A, 18B that define the groove 17 there between. At the most distal end of the temple portion 14 of the eyewear, the legs 18A, 18B converge to form a separable engagement 27 that is biased in a closed position. In other words, the legs 18A, 18B of the temple portion 14 are shaped such that the outermost distal end of the temple portion 14 forms an entryway 27 for the coupling element 21 described above. The shape of the legs 18A, 18B and the position of the legs on either side of the groove 17 in the temple 19 bring the legs together at the distal end. The legs 18A, 18B touch but are separable by a force that pries the legs apart. This configuration is adapted for receiving the coupling element 21 of the hair band 10 through the entryway 27 to the groove 17 in the temple portion 19.

When attaching the eyewear shown in FIG. 7 to the hair band assembly of FIG. 1, each temple portion 19 of the eyewear slides into the cavity 24 defined by the hair band 10. The coupling element 21 in the cavity engages the closed leg portions 18A, 18B and pries the legs apart. The coupling element slides into the groove 17 in the temple portion 19 and allows for sizing of the eyewear to different wearers. The shape of the groove 17 includes a divot 28 in which the coupling element 21 may initially rest unless moved by the wearer to a different position along the groove 17. This divot 28 provides an initial resting position that may serve as the default position for the coupling element in the groove. Again, the default position in the divot is arranged for the most often used size of eyewear, but the groove allows for the temple portion, and therefore the entire frame, to be adjusted lengthwise for wearers who desire a different length along the temple.

The divot 28 is also useful in allowing the coupling element 21 to rotate within the groove 17, so that the frame element 14 can be retracted into the cavity of the hair band. In this regard, the divot along the groove is a pivoting point for the coupling element, as it gives more leeway for rotation about the axis provided by the coupling element.

The coupling element is sized to fit within the groove such that it maintains a stable position that requires an outside force for adjustment. The coupling element is sufficiently secured within the cavity of the hair band to withstand the force of prying the legs of the temple portion apart.

In addition to adding pivoting capabilities, the coupling element and groove 17 or slide rail 21 enable the eyeglasses frame element 14 to be fitted to a wearer's head and face. In other words, the coupling element 20 slides along the slide rail 21 or the groove 17 (or, vice versa, the slide rail slides along the coupling element 20) so that the distance between the coupling element 20 and the far edge of the frame element (proximate the lenses) can vary. In this way, a single hair band 10 fits individuals with varying head sizes.

As shown in FIG. 4, the slide rail 21 also includes an indentation 26, which is the default location for the coupling element 20 within the slide rail 21. The default location within the indentation 26 is the most convenient location for sizing the frame element 14 so that it fits within the cavity 24. In a preferred use, a wearer would raise up the frame element 14 from an extended position on the wearer's face, size the frame element 14 to the default location within the indentation 26, and then slide the frame element 14, including lenses into the cavity 24. All of these steps occur without removing the hair band 10 from the wearer's head. Accordingly, the depiction of the indentation 26 within the slide rail 21 in FIG. 4 is illustrative only and is not intended to show the only mechanism for fitting the frame element 14 into the cavity 24. The indentation 26 may be located at any convenient location within the slide rail 21 to allow the frame element 14 to retract up into the cavity 24. The indentation does not prohibit any back and forth movement of the frame element, but allows the user to find the best spot for sizing the frame element so that it fits within the cavity 24. The coupling element 20 fits nicely within the indentation 26 and pops into place to allow the user to know that the frame element 14 is in a position ready for retracting into the cavity 24. The coupling element 20, however, still allows the frame element 14 to slide back and forth along the slide rail 21 with minimal force into and out of the indentation 26.

In addition to coupling the frame element 14 to the hair band 10, the coupling element 20 may connect the upper band element 11 to the lower band element 12. Alternatively, the band elements 11 and 12 may be connected by any other appropriate means that does not interfere with the structure or operation of the hair band 10. For example, the upper and lower band elements 11, 12 may be connected along various edges of the band elements so long as the connection points of the band elements 11, 12 do not interfere with the operation of the overall hair band 10 assembly.

The upper band element 11 and the lower band element 12 define a cavity 24, in which the frame element 14 may be positioned when retracted. FIG. 6 depicts a zoomed in cross-sectional view of the cavity 24. FIG. 4, a cross-sectional view of the hair band 10, depicts the frame element 14 being in the retracted position.

In a preferred embodiment, the frame element 14 also includes a protrusion 16 and a glide tab 15. The upper band portion has a protrusion snap lock 23 complementary to the protrusion 16 for securing the frame element 14 when the frame element 14 is in the cavity position. The upper band portion 11 also has a recess 13 positioned so that the glide tab 15 fits into the recess 13 when the frame element 14 is in the retracted position.

The combination of the glide tab 15 and the recess 13 provides a manual cue to the wearer regarding the location of the most convenient portion of the frame element 14 that the wearer should grasp to pull the frame element 14 out of the cavity 24. When the frame element 14 is in the retracted position, the glide tab 15 mates with the recess 13 to create a substantially uniform surface along the front edge, or ridgeline 25, of the upper band element 11. The uniformity is aesthetically appealing, but the wearer still has the ability to detect a difference in the surface of the front edge 25 of the upper band element 11 even when the frame element 14 is in the retracted position. That difference manually alerts the wearer to pull down on the glide tab 15 to move the frame element 14 into an extended position over the wearer's eyes.

As noted above, the frame element 14 includes a raised bump, or protrusion 16, on the outer region of the frame element 14. This protrusion 16 slides between upper and lower bands 11, 12 such that the top of the protrusion 16 is adjacent the underside of the upper band 11. In other words, the protrusion 16 slides into the cavity 24 between the frame element 14 and the upper band element 11. The upper band element 11 defines a recess, referred to herein as a protrusion snap lock 23, on its underside adjacent the cavity 24. FIG. 4 depicts the protrusion 16 being mated to the protrusion snap lock 23. The protrusion snap lock 23 engages the protrusion 16 with a sufficient hold to releasably maintain the frame element 14 in the cavity 24 as desired. Of course, the overall hair band assembly may include a pair of protrusions 16 and a pair of protrusion snap locks 23. A respective pair 16, 23 may be located on either side of the frame element 14 and hair band 10 combination.

FIG. 4 further shows the glide tab 15 being mated to the recess 13. The glide tab 15 enables the wearer of the hair band 10 to grip the frame element 14 so that it may be pivoted from the refracted position inside the cavity 24 to the extended position on the wearer's face.

FIG. 5 is a front elevational view of the hair band 10. FIG. 5 depicts a plane 30 bisecting the hair band 10. The upper band element 11 has a top portion 31 defined by the intersection of the upper band element 11 with the plane 30. The lower band element 12 has a top portion 33 defined by the intersection of the lower band element 12 with the plane 30. In a preferred embodiment, the top portion 31 of the upper band element 11 is wider than the top portion 33 of the lower band element 12 so that a front edge, or ridgeline, 25 substantially obscures the frame element 14 when the frame element 14 is in the cavity 24 position. The upper band element 11 also has two tip portions 32. The upper band element 11 may be tapered from the top portion 31 of the upper band element to each of the tip portions 32 of the upper band element. The lowered band element 12 may be similarly tapered.

The hair band 10 may further include one or more decorative elements. One or more connectors may be on the upper band element 11 for connecting and readily removing one or more of the decorative elements to the upper band portion 11.

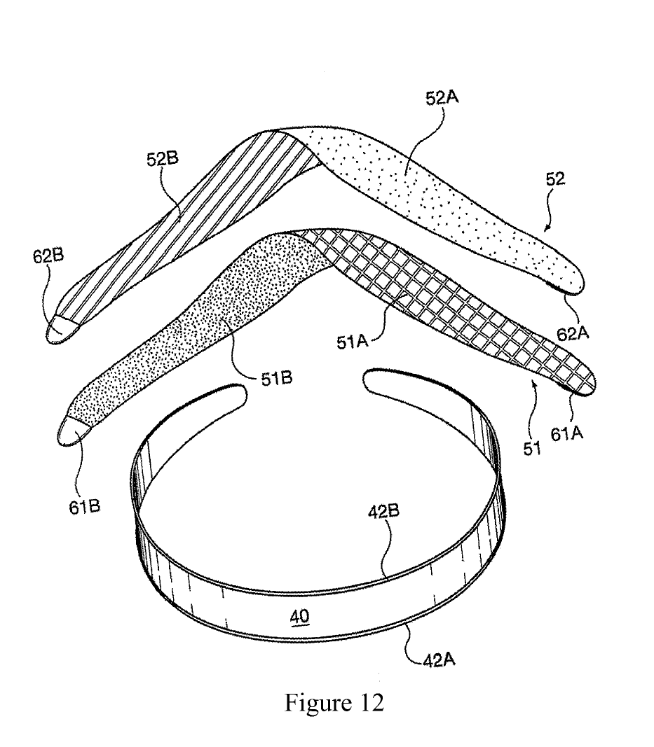

The removable decorative elements are shown in more detail in FIG. 12. FIG. 12 shows a hair band 40 similar to the hair band 10 described above and of the same general shape. The hair band 40 includes optional retaining guides 42A and 42B extending along the arcuate edges of the hair band. The optional retaining guides 42A and 42B may be described as raised lips on one edge or both edges of the hair band. Removable decorative elements 51 and 52 are designed to fit on the hair band 40 and stay in place by extending across the hair band between retaining guides 42A and 42B. For hair bands with only one retaining guide, the decorative element 51, 52 fits alongside the retaining guide.

The removable decorative elements of FIG. 12 are shown as two different strips of fabric that are reversible. The removable and reversible decorative elements 51, 52 have different prints on either side of the fabric. For decorative element 51, a first side 51A of the decorative element 51 is a pattern of checks and the underside 51B of the decorative element 51 is a densely dotted pattern. A second decorative element 52 also has two sides for alternative use with the hair band 40. A top side of decorative element 52 has sparsely dotted pattern, and the second side of decorative element 52 has a striped pattern. The types of patterns are examples only and in no way limit the invention, but the decorative elements have a reversible functionality such that one strip of fabric has two sides that can be used with the hair band for different looks. The reversible nature of the decorative elements 51, 52 gives the user more options for matching the outer side as desirable with different outfits.

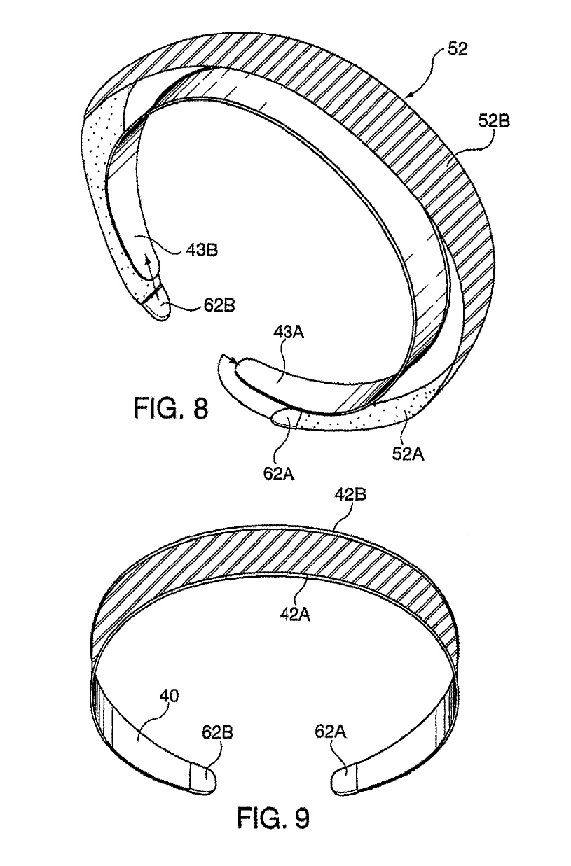

One useful feature of the decorative elements 51, 52 is the pocket tab used to hold a decorative element 51, 52 onto the hair band 40. Each decorative element 51, 52 has a pair of pocket tabs 61A, 61B, 62A, 62B on each respective end of the decorative element. The pocket tabs fit around a respective end of the hair band 40 to hold the decorative element in place on the hair band 40 alongside and/or between retaining guides 42A, 42B. FIG. 8 shows decorative element 52 fitting around hair band 40. Respective ends 43A, 43B of the hair band 40 fit within pocket tabs 62A, 62B on the decorative element. FIG. 9 shows decorative element 52 in place around the hair band 40.

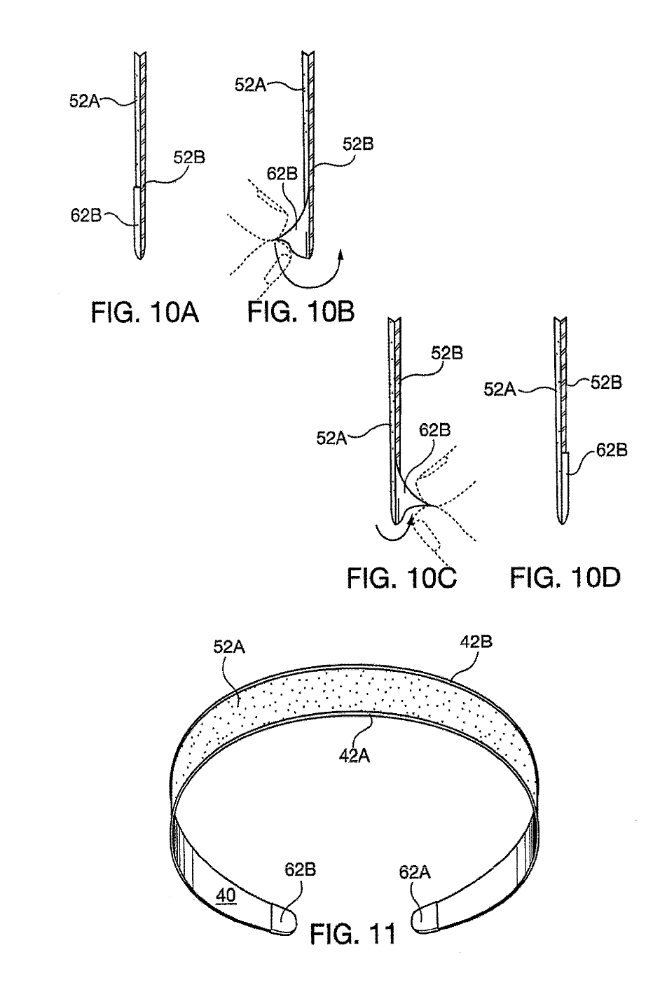

For a more detailed view of the reversible nature of the decorative elements 51, 52, FIGS. 10A-10D illustrate the concept of how a single pocket tab on either end of the decorative element can be used on either reversible side of the decorative element. Starting with FIG. 10A, end 43B of the hair band 40 fits within pocket tab 62B such that one side 52A of the decorative element would be worn directly adjacent the hair band and the other side 52B would be exposed. The pocket tab 62B is allowed to be flipped inside out as in FIGS. 10B and 10C so that the same pocket tab 62B is adjacent the other side of the decorative element 52B. In this way, the same hair band 40 fits within the pocket tab 62B such that decorative element side 52B is on the interior and directly adjacent the hair band 40. In the drawing of FIG. 10D, the pocket tab has been flipped inside out so that side 52A could be worn on the outside of the hair band 40. FIG. 11 shows this embodiment installed on the hair band 40.

In the specification and drawings, typical embodiments of the invention have been disclosed and, although specific terms have been employed, they have been used in a generic and descriptive sense only and not for purposes of limitation. Different kinds of materials and elements may be substituted for the parts disclosed herein, and any method steps can be adjusted yet still fall within the ambit of the invention. The invention is further set forth in the claims below.

1. A hair band assembly comprising:
a hair band defining a cavity between opposite ends of the hair band, wherein the cavity is accessible from one side of the hair band; and
a pair of eyeglasses that fit within the cavity of the hair band.

2. A hair band assembly according to claim 1, further comprising a coupling element within the cavity, said coupling element engaging both the eyeglasses and the hair band, wherein said eyeglasses pivot about said coupling element between a retracted position within the cavity and an extended position on the wearer's face.

3. A hair band assembly according to claim 1, wherein said eyeglasses comprise legs that define a groove within a temple portion of the eyeglasses, said legs being separable at one end.

4. A hair band assembly according to claim 3, further comprising a coupling element within the cavity of the hair band, said coupling element engaging the hair band and fitting between the legs of the temple portion of the eyeglasses such that that coupling element slides within the groove.

5. A hair band assembly according to claim 4, wherein said coupling element is removable from the groove of the temple portion and the eyeglasses are separable from the hair band.

6. A hair band assembly according to claim 1, further comprising a removable decorative element on the hair band.

7. A hair band assembly according to claim 6, wherein said decorative element comprises a fabric having a reversible pocket tab on the decorative element that attaches to an end of the hair band to hold the decorative element in place but allowing the decorative element to be removed.

8. A hair band comprising:
an upper band element, wherein said upper band element is shaped as an open loop that conforms to a head of a wearer when positioned over the top of the wearer's head;
a lower band element connected to said upper band element, wherein said lower band element is shaped as an open loop that conforms to the head of the wearer when positioned over the top of the head, and wherein said upper band element and said lower band element define a cavity there between;
a frame element for extending around a wearer's face;
a coupling element for connecting said frame element to at least one of said band elements, wherein said coupling element enables said frame element to be pivoted between an extended position on a wearer's face and a retracted position in the cavity of the hair band.

9. A hair band according to claim 8 wherein said frame element defines a slide rail for engaging said coupling element.

10. A hair band according to claim 9 wherein the position of said coupling element along said slide rail is adjustable.

11. A hair band according to claim 8 wherein:
said upper band element defines a recess; and
said frame element defines a glide tab which fits into said recess for allowing said frame element to be readily pivoted from said cavity position to said extended position.

12. A hair band according to claim 8 wherein:
said frame element defines a protrusion; and
said upper band element defines a protrusion snap lock substantially complimentary to said protrusion for securing said frame element when said frame element is in said retracted position within the cavity.

13. A hair band according to claim 8 further comprising:
a decorative element on said hair band; and
at least one connector on said decorative element for connecting and readily removing said decorative element to the hair band.

14. A hair band according to claim 1 wherein said band elements are tapered.

15. A hair band and decorative element combination comprising
a reversible pocket tab on the decorative element that attaches to an end of the hair band to hold the decorative element in place but allowing the decorative element to be removed.

16. A hair band and decorative element combination according to claim 15, wherein said hair band further comprises at least one retaining guide extending along the arcuate edges of the hair band.

17. A hair band and decorative element combination according to claim 15, wherein said decorative element is reversible.

18. A hair band and decorative element combination according to claim 15, wherein said pocket tab is sufficiently flexible for flipping inside out, thereby allowing an end of the hair band to be situated therein.

19. A hair band and decorative element combination according to claim 18, wherein the pocket tab is moveable to display either side of the decorative element on the outside of the hair band.

20. A hair band and decorative element combination according to claim 15, wherein the hair band defines a cavity between opposite ends of the hair band, and the cavity is accessible from one side of the hair band; the hair band further comprising a pair of eyeglasses that fit within the cavity of the hair band.
